(19) United States
(12) Patent Application Publication
(10) Pub. No.: US 2024/0167973 A1
(43) Pub. Date: May 23, 2024

(54) DIGITAL CIRCUIT ARRANGEMENT FOR CHEMIRESISTIVE GAS SENSORS

(71) Applicant: King Faisal University, Al-Hassa (SA)

(72) Inventors: Nagih Mohammed Shaalan, Al-Hassa (SA); Ahmed Mohamed Mossad, Al-Hassa (SA)

(21) Appl. No.: 17/990,030

(22) Filed: Nov. 18, 2022

(51) Int. Cl. G01N 27/12; G01N 33/00; G01N 33/02

(52) U.S. Cl. CPC G01N 27/125; G01N 33/0006; G01N 33/025

(57) ABSTRACT

A device, method, and system having a gas sensor, such as those in used in electronic noses or e-noses, for gas detection in a sample. In particular, the device, method, and system is concerned with ethylene gas detection for monitoring of the ripening of fruit.

### BACKGROUND

### 1. Field

This disclosure is directed towards providing efficient and reliable gas sensors, such as those in used in electronic noses or e-noses, for various purposes. In particular, this disclosure is concerned with ethylene gas detection for the monitoring of the ripening of fruit.

### 2. Description of the Related Art

The global gas sensor mark was valued at 2.5 billion in 2021 and is expected to expand at a compound annual growth rate (CAGR) of 8.9% from 2022 to 2030. The main factor in driving the gas sensor market is an advancement in the miniaturization of components, along with the improvement in communication technologies that enable the sensors to be integrated into various devices and machines to detect toxic gases remotely at a safe distance. The agricultural sector captured a share of about 10% of the global revenue of the gas sensing market for 2021. Ethylene gas sensors are a particular type of gas sensor which heretofore have been cost prohibitive and are not sufficiently accurate for their intended purposes.

In this regard, CN 111077099 A discloses a formaldehyde concentration detection method and device based upon infrared absorption spectra which simultaneously detects the positions of two infrared absorption peaks of formaldehyde gas to detect the absorption condition of formaldehyde to infrared light, and finally calculate the formaldehyde concentration. Because the gas in the atmosphere has a characteristic absorption peak and cannot be superposed with the two absorption peaks of the formaldehyde, the influence of the other gases can be greatly reduced by selecting the unaffected absorption peaks as the data of the detected concentration.

Similarly, JP 41550803 B2 is directed towards a gas sensor for measuring gas concentration. A semiconductor gas sensor indirectly heats a semiconductor material that is sensitive to gas by a heating element. A known resistor is connected to the semiconductor material and is adjusted to a plurality of predetermined values. A power supply supplies a driving voltage to the semiconductor material and the known resistor. The load voltage of the known resistor is A/D convened into a digital signal. Stored calibration curve data is obtained and a gas concentration calculator uses a difference between a resistance ratio in the calibration curve with respect to a known concentration and resistance ratio for each sensor is set as a coefficient, and the gas concentration is calculated accordingly.

In addition, U.S. Pat. No. 10,145,828 B2 discloses a system and method for automatically adjusting gas sensor settings and parameters. An automatic sensor excitation voltage adjustment feature, a multi-range concentration feature, a single calibration feature, and a barrier circuit are all disclosed. The automatic sensor excitation voltage adjustment feature includes a transmitter having a transmitter microprocessor that provides an initial voltage to a sensor having a sensor microprocessor. As the voltage changes a correction signal is relayed from the sensor microprocessor to the transmitter microprocessor. The correction signal is used to adjust the voltage applied to the sensor. The multi-range concentration sensor feature includes an amplifier associated with the sensor/microprocessor to create gain settings used to optimize sensor resolution by changing a gain value for the sensor.

Further, U.S. Pat. No. 11,078,020 B2 discloses a system and method for ripening produce. A ripening schedule for produce is created and the ripening schedule is implemented at a ripening chamber as an effective tool to control the environmental conditions and the time spent in the ripening chamber by the produce in order to conform ripening conditions of the produce to the target shipping date. The ripening schedule is applied to control ripening conditions in the ripening chamber. The system and method make use of a plurality of gas sensors to ensuring that the ripening chambers are configured to produce fruit at an optimal condition for display in stores.

However, all of these known systems and methods are overly complicated and/or expensive when, for example, measuring ripeness of fruit. What is needed is an accurate, easy to use, and inexpensive device to accurately determine fruit ripeness and quality, thereby reducing spoilage of the produce while in transit to grocery stores and while on display at grocery stores. Customers are much more likely to purchase fruit or produce that is appealing to the eye and in otherwise optimal condition for immediate consumption.

### SUMMARY

The device, system, and method as discussed herein detect and monitor the presence of certain gases in a sample. By way of non-limiting example, the present device, system, and method can detect ethylene gas as it is emitted from aging fruit or produce. The detection occurs through the use of gas sensors configured as an electronic nose (an ‘e-nose’) which can detect miniscule amounts of ethylene gases accurately in parts per billion (ppb). The e-nose sensor is connected to other associated digital circuitry, which performs an A/D conversion of the detected signal and communicates that information to a microcontroller, which processes the information to perform sensor calibration. The circuit determines a correlation between a voltage difference and a gas ratio in ppm and then maintains that correlation through multiple uses of the gas sensor to detect the particular gas to which the sensor has been calibrated.

In one embodiment, the present subject matter relates to a chemiresistive device for detecting gases contained within a housing, comprising: a) a chemiresistive gas sensor; b) an A/D converter connected to the chemiresistive gas sensor; c) a microcontroller connected to the chemresistive gas sensor; d) a display connected to the microcontroller; e) a keypad connected to the microcontroller; and f) a voltage converter connected to the keypad, the chemiresistive gas sensor, the A/D converter, the keypad, the microcontroller, and the display.

In an embodiment, the chemiresistive device for detecting gases can further comprise a power supply located within the housing for providing an input voltage to the voltage converter. In one embodiment in this regard, the housing can be a hand-held housing that includes any one or more, or all, of the display, the keypad, a ground pin, a signal port analog pin, and a 5-volt pin. In an alternative embodiment, the chemiresistive device for detecting gases can be connected to an external power supply for providing an input voltage to the voltage converter.

In another embodiment, the present subject matter relates to a method for detecting gases using a chemiresistive device, comprising: a) calibrating the chemiresistive device for detecting gases, wherein the calibration provides voltage values against known concentrations of known gases, determines a correlation between the voltage values and the known concentrations of known gases in parts per million (ppm), and stores a plurality of said voltage values from first and second ends of the chemiresistive gas sensor against a plurality of said known gas concentrations of said known gases, and the correlations thereof; b) placing the chemiresistive device for detecting gases in contact with a gas sample; c) measuring a voltage value of a gas sample; d) performing an A/D conversion of the voltage value of the gas sample; e) displaying a graph of the A/D converted voltage value of the gas sample; f) comparing the voltage value of the gas sample to the plurality of voltage values of the plurality of known gas concentrations of the known gases and the correlations thereof; and g) determining whether the gas sample contains a specific concentration in ppm or fraction of ppm.

These and other features of the present subject matter will become readily apparent upon further review of the following specification.

### BRIEF DESCRIPTION OF THE DRAWINGS

FIG. 1 is a view of the e-nose sensor in a voltage divider circuit.

FIG. 2 is a diagram of the e-nose sensor and connected circuitry.

FIG. 3 is a keypad connected to the e-nose sensor and connected circuitry.

FIG. 4 is a flowchart of the operation of the device.

Similar reference characters denote corresponding features consistently throughout the attached drawings.

### DETAILED DESCRIPTION OF THE PREFERRED EMBODIMENTS

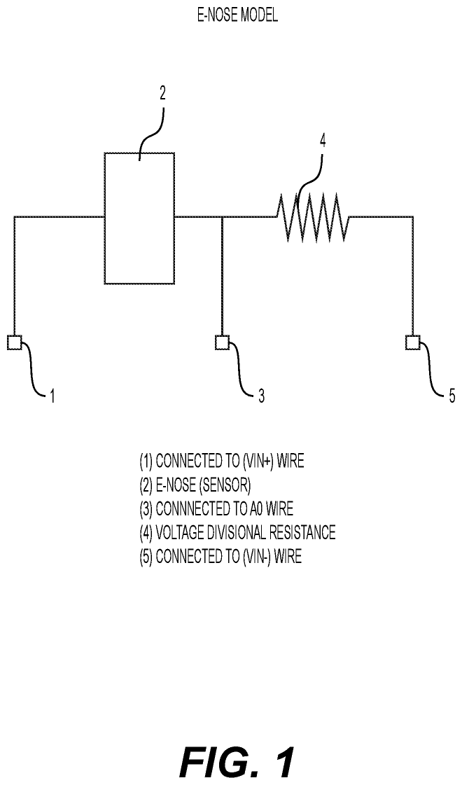

The digital circuit arrangement of a chemiresistive device for detecting gases as described herein is constructed in several parts. As shown in FIG. 1, the chemiresistive gas sensor of the present devices includes a sensor, by way of non-limiting example, an electronic nose, or e-nose, gas sensor component (2), a voltage dividing resistance (4), two voltage input wires, a VIN+ (1), a VIN− (5), and an analog output wire (3).

In this regard, an “electronic nose” or “e-nose” gas sensor can be used interchangeably herein to refer to a sensor that can detect the change that occurs in its voltage values as a result of a change in its resistance, to indicate the gas concentration across a programmed microcontroller.

The gas sensor component (2) is a multi-gas sensor and can be calibrated to a particular gas, as desired. In one non-limiting example in this regard, the gas sensor component (2) can be an ethylene gas sensor for detecting miniscule amounts of ethylene gas in the parts per million (ppm) range. The digital circuit of the ethylene gas sensor is mainly based on an A/D converter circuit which measures the potential difference across an adjustable voltage divider circuit (4) between opposite first and second ends of the e-nose gas sensor. By way of non-limiting example, the A/D converter can be a 16-bit A/D converter. In this regard, the adjustable voltage divider circuit can provide a voltage value during a calibration of the electronic nose gas sensor or e-nose gas sensor against a known concentration of a known gas, as well as provide voltage values in the form of voltage drops between the first end and the second end of the electronic nose gas sensor or e-nose gas sensor when measuring for the presence of a gas in a sample.

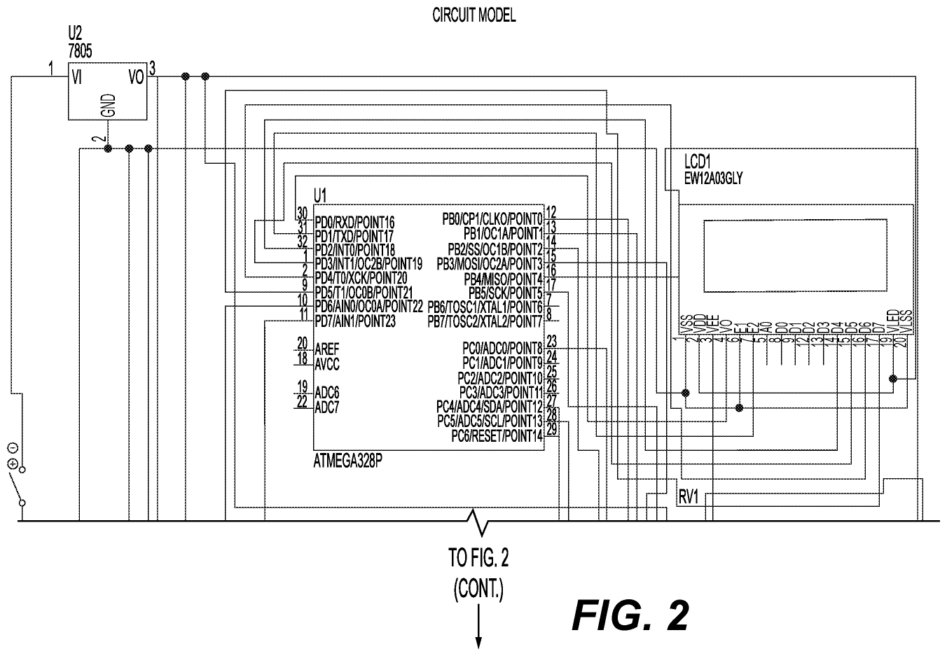

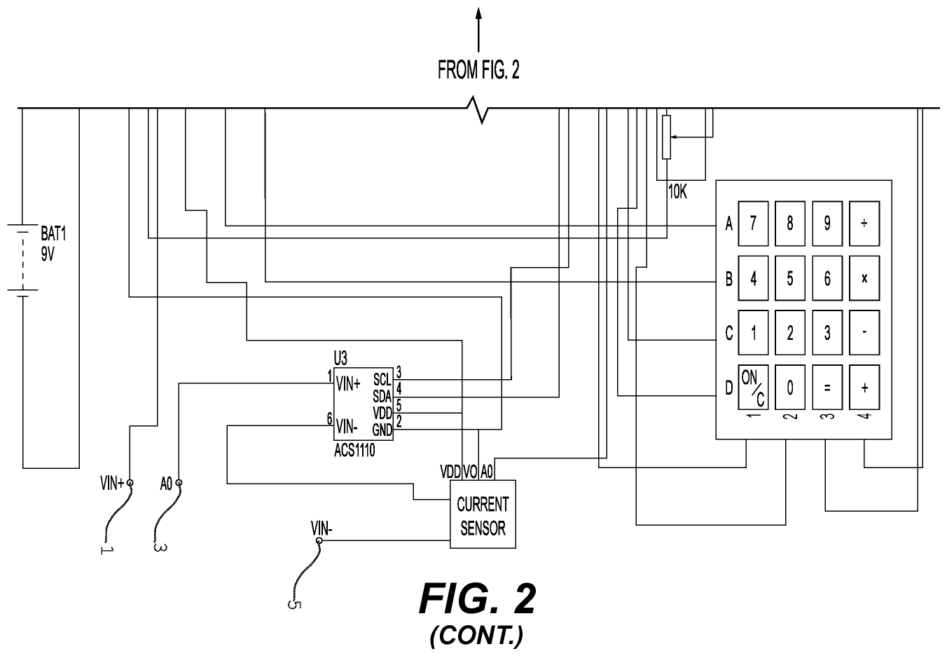

A circuit model of the device is shown in FIG. 2 and shows the voltage divider circuit (4) of FIG. 1 being connected to a display screen (LCD1) and a microcontroller (U1) with a plurality of signal and power lines. As shown in FIG. 2, the microcontroller (U1) has a plurality of twenty-seven input and output lines which connect to a display screen (LCD1), a voltage converter (U2), a 9V power supply (BAT1), the aforementioned sixteen bit A/D converter (U3), a chemiresistive ethylene gas sensor, and a keypad, such that the microcontroller controls the interaction of the display screen, keypad, A/D converter, 9V power supply, and gas sensor with each other during the ethylene gas sensor calibration process and the subsequent testing procedure of the fruit or produce in question.

Based upon the measured potential difference of the voltage drop across the voltage divider circuit (4), the microcontroller (U1) can cause the display screen (LCD1) to display a voltage graph of the gas sample. The circuit model of the device can include a keyboard, which can be used during manual sensor calibration to manually key in the gas concentration of the gas sample in parts per million (ppm). It is then possible to record multiple voltage values on the e-nose against a corresponding gas concentration so that the device can find the relationship between the voltage values and the various gas concentrations and determine a ratio between them. In one embodiment, it is possible to record up to eight voltage values in this regard. The microcontroller memorizes the ratio in its onboard internal memory such that this stored ratio can be used on-site to find a particular ethylene gas concentration so that calibration does not need to be performed on-site and the device can be applied strictly towards measurement procedures, thereby decreasing costs and time involved in the process.

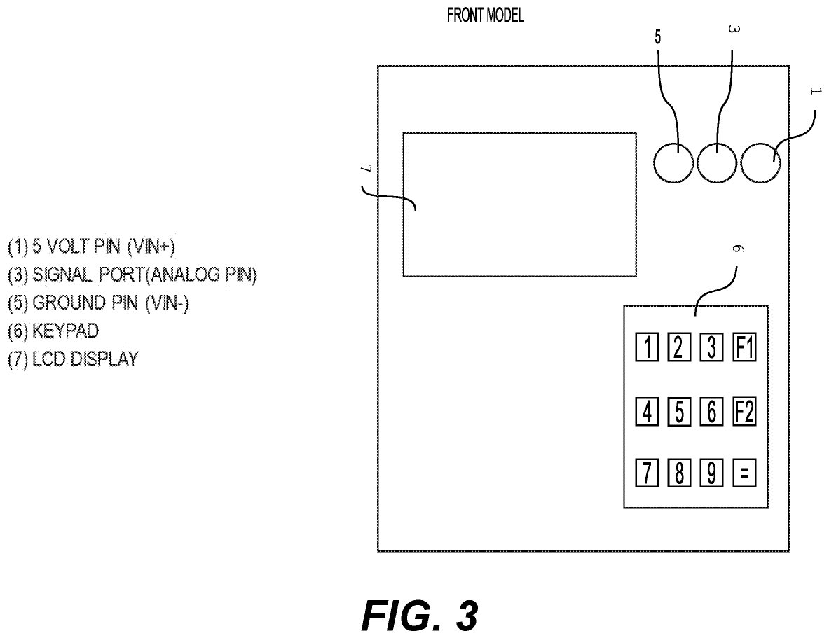

As shown in FIG. 3, a front view of the device housing is depicted showing a keypad (6), the aforementioned LCD screen (7) and various pins/ports (1), (3), (5). In one embodiment, this device is a hand-held device and is therefore easily transportable to various on site locations by the user.

In one embodiment, the circuit model of the present device is specifically designed to monitor the ripening of fruits and produce, in particular, bananas, by detecting for the presence and concentration of ethylene gas in a sample. However, the chemiresistive gas sensor can be used to detect any chemiresistive resistance presence as the voltage divider can be adjusted according to the desired resistance range of the sensor. The range of adjustment can be from a few ohms to mega ohms.

In one embodiment of the circuit model of the device, the chemiresistive gas sensor operates in the range of kilo ohms to mega ohms, for example, to detect and measure the presence and concentration of ethylene gas in a sample. In this regard, the adjustable voltage divider circuit can have an adjustable resistance with an adjustable resistance range of 1 kilo ohm to 1 mega ohm, as well as any values therebetween. Additionally, the chemiresistive gas sensor can operate accurately at room temperature or higher and is therefore very sensitive to a low change in e-nose resistance. The e-nose sensor is very sensitive at low temperatures. In one embodiment, the e-nose can be fabricated in a gold layer. One example of such a device was in-situ tested for detection of different ethylene gas sensors and was tested against a MQ3 type alcohol detection sensor. After the manufacturing process of the circuit and chemiresistive gas sensor, a calibration procedure can be performed such that the sensor arrives to the user already factory calibrated. However, if the user desires to calibrate the gas sensor themselves that function is possible as well.

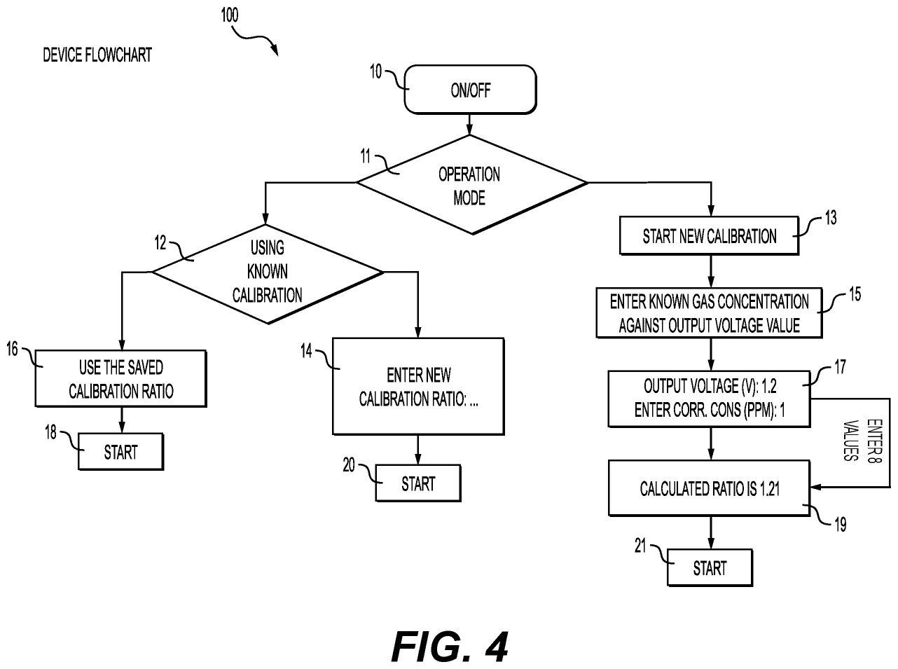

As shown in FIG. 4, the flowchart of the device outlines the calibration process (100) which a user can initiate as needed. After turning on the device (10), the process proceeds to an operation mode decision block (11) where the user can rely on a known factory calibration (12) or start a new on-site calibration (13). In this regard, the calibration step can be earlier completed at a site remote from where the chemiresistive device for detecting gases is placed in contact with the gas sample, or can be completed on site immediately prior to the chemiresistive device for detecting gases being placed in contact with the gas sample.

If a known calibration is chosen, the user can either select the usage of a known and already saved calibration ratio (16) or enter by means of the keypad a new preferred calibration ratio (14). Either calibration ratio selection mode leads to its own respective start block for actual measurement (18) and (20). If the user eschews the factory calibration and desires a new calibration (13), the user then measures a known gas concentration against a measured output voltage value (15) and enters the corresponding gas concentration values (17) and an additional seven pairs of values, to calculate an on-site ratio (19). One satisfied with the calibration process, the user can proceed to an additional start block for actual measurement (21).

It is to be understood that the present device, system, and method for chemiresistive gas sensor calibration and measurement is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A chemiresistive device for detecting gases contained within a housing, comprising:
a) a chemiresistive gas sensor;
b) an A/D converter connected to the chemiresistive gas sensor;
c) a microcontroller connected to the chemiresistive gas sensor;
d) a display connected to the microcontroller;
e) a keypad connected to the microcontroller; and
f) a voltage converter connected to the keypad, the chemiresistive gas sensor, the A/D converter, the keypad, the microcontroller, and the display,
wherein the display is an LCD screen capable of displaying a voltage graph curve,
wherein said chemiresistive gas sensor is an electronic nose gas sensor or e-nose gas sensor,
wherein said electronic nose gas sensor or e-nose gas sensor is fabricated in a gold layer, and
wherein said electronic nose gas sensor or e-nose gas sensor includes an adjustable voltage divider circuit between a first end of the electronic nose gas sensor or e-nose gas sensor and a second end of the electronic nose gas sensor or e-nose gas sensor, wherein said adjustable voltage divider circuit provides a voltage value during a calibration of the electronic nose gas sensor or e-nose gas sensor against a known concentration of a known gas, and wherein during said calibration of the electronic nose gas sensor or the e-nose gas sensor, the microcontroller stores a plurality of voltage values from the first and second ends of the electronic nose gas sensor or the e-nose gas sensor against a plurality of known gas concentrations.

2. The chemiresistive device for detecting gases as recited in claim 1, further comprising a power supply for providing an input voltage to the voltage converter.

3-6. (canceled)

7. The chemiresistive device for detecting gases as recited in claim 1, wherein said microcontroller can cause said display to display a graph of a voltage value of a gas sample after the electronic nose gas sensor or the e-nose gas sensor takes a measurement of the gas sample based on a measured potential difference of voltage drop across the adjustable voltage divider circuit.

8. The chemiresistive device for detecting gases as recited in claim 7, wherein said housing is a hand-held housing and further includes the display, the keypad, a ground pin, a signal port analog pin, and a 5-volt pin.

9. The chemiresistive device for detecting gases as recited in claim 1, wherein said adjustable voltage divider circuit has an adjustable resistance with an adjustable resistance range of 1 kilo ohm to 1 mega ohm.

10. The chemiresistive device for detecting gases as recited in claim 1, wherein the electronic nose gas sensor or the e-nose gas sensor is operative at room temperature.

11. The chemiresistive device for detecting gases as recited in claim 1, wherein the electronic nose gas sensor or the e-nose gas sensor, once calibrated, detects and measures ethylene gas.

12. The chemiresistive device for detecting gases as recited in claim 11, wherein the electronic nose gas sensor or the e-nose gas sensor, once calibrated, detects and measures ethylene gas in order to ascertain ripeness in produce and fruit.

13. The chemiresistive device for detecting gases as recited in claim 12, wherein said fruit is a banana.

14. The chemiresistive device for detecting gases as recited in claim 12, wherein the electronic nose gas sensor or the e-nose gas sensor are capable of detecting and measuring the ethylene gas accurately in parts per million (ppm).

15. A method for detecting gases using a chemiresistive device, comprising:
a) calibrating the chemiresistive device for detecting gases as recited in claim 1, wherein the calibration provides voltage values against known concentrations of known gases, determines a correlation between the voltage values and the known concentrations of known gases in parts per million (ppm), and stores a plurality of said voltage values from first and second ends of the chemiresistive gas sensor against a plurality of said known gas concentrations of said known gases, and the correlations thereof;
b) placing the chemiresistive device for detecting gases in contact with a gas sample;
c) measuring a voltage value of a gas sample;
d) performing an A/D conversion of the voltage value of the gas sample;
e) displaying, using an LCD screen, a voltage graph curve of the A/D converted voltage value of the gas sample;
f) comparing the voltage value of the gas sample to the plurality of voltage values of the plurality of known gas concentrations of the known gases and the correlations thereof; and
g) determining whether the gas sample contains a specific gas.

16. The method of claim 15, wherein the calibration step can be earlier completed at a site remote from where the chemiresistive device for detecting gases is placed in contact with the gas sample, or can be completed on site immediately prior to the chemiresistive device for detecting gases being placed in contact with the gas sample.

17. The method as recited in claim 15, wherein said chemiresistive gas sensor is an electronic nose gas sensor or e-nose gas sensor.

18. The method as recited in claim 17, wherein the electronic nose gas sensor or the e-nose gas sensor, once calibrated, is configured to detect and measure ethylene gas in order to ascertain ripeness in produce and fruit.

19. The method as recited in claim 18, wherein said fruit is a banana.

20. The method as recited in claim 17, wherein the electronic nose gas sensor or the e-nose gas sensor, once calibrated, maintains the correlation through multiple uses of the gas sensor to detect the particular gas to which the sensor has been calibrated.
